(19) United States
(12) Patent Application Publication
(10) Pub. No.: US 2009/0325986 A1
(43) Pub. Date: Dec. 31, 2009

(54) Deazapurine Analogs of 1'-Aza-L-Nucleosides

(76) Inventors: Richard Hubert Furneaux, Wellington (NZ); Peter Charles Tyler, Wellington (NZ); Gary Brian Evans, Lower Hutt (NZ); Vern L. Schramm, New Rochelle, NY (US); Keith Clinch, Lower Hutt (NZ)

Correspondence Address: AMSTER, ROTHSTEIN & EBENSTEIN LLP, 90 PARK AVENUE, NEW YORK, NY, 10016, US

(21) Appl. No.: 12/086,134

(22) PCT Filed: Dec. 15, 2006
(86) PCT No.: PCT/NZ2006/000331

§ 371 Date: Aug. 25, 2009

(30) Foreign Application Priority Data
Dec. 15, 2005 (NZ) 544187

(51) Int. Cl. A61K 31/519; C07D 487/04; A61P 37/00; A61P 33/00; A61P 35/00

(52) U.S. Cl. 514/265.1; 544/280

(57) ABSTRACT

The invention relates to compounds of the formula (I), which are L-enantiomeric forms of nucleoside analogues, and to pharmaceutical compositions containing the compounds, methods of treating certain diseases, including cancer, bacterial infection, parasitic infection, and T-cell mediated diseases, using the compounds, processes for preparing the compounds, and intermediates useful in the preparation of the compounds.

### TECHNICAL FIELD

This invention relates to certain L-enantiomeric forms of nucleoside analogues, the use of these compounds as pharmaceuticals, pharmaceutical compositions containing the compounds, methods of treating certain diseases using the compounds, processes for preparing the compounds, and intermediates useful in the preparation of the compounds.

### BACKGROUND

Recent research in the area of purine nucleoside phosphorylase (PNP), methylthioadenosine phosphorylase (MTAP) and 5′-methylthioadenosine nucleosidase (MTAN) and nucleoside hydrolase inhibitors has resulted in the design of a class of compounds known as the Immucillins, some of which are potent inhibitors of one or more of the above enzymes. Immucillins are nucleoside analogues where the sugar has been replaced with an imino sugar moiety.

PNP catalyses the phosphorolytic cleavage of the ribo- and deoxyribonucleosides of guanine and hypoxanthine to give the corresponding sugar-1-phosphate and guanine or hypoxanthine.

Humans deficient in PNP suffer a specific T-cell immunodeficiency due to an accumulation of dGTP and its toxicity to stimulated T lymphocytes. Because of this, inhibitors against PNP are immunosuppressive, and are active against T-cell malignancies.

U.S. Pat. No. 5,985,848, U.S. Pat. No. 6,066,722 and U.S. Pat. No. 6,228,741 describe compounds that are inhibitors of PNP and purine phosphoribosyltransferases (PPRT). The compounds are useful in treating parasitic infections, T-cell malignancies, autoimmune diseases and inflammatory disorders. They are also useful for immunosuppression in organ transplantation.

U.S. Pat. No. 6,693,193 describes a process for preparing certain PNP inhibitor compounds, providing another useful route to the synthesis of this class of compounds. U.S. Pat. No. 7,109,331 discloses further compounds that are inhibitors of PNP and PPRT.

The imino sugar part of the inhibitor compounds referred to above (generally known as Immucillins) has the nitrogen atom located between C-1 and C-4 so as to form 1,4-dideoxy-1,4-imino-D-ribitol compounds. The location of the nitrogen atom in the ribitol ring may be important for binding to enzymes. In addition, the location of the link between the imino sugar moiety and the nucleoside base analogue may be critical for enzyme inhibitory activity. The compounds described above have that link at C-1 of the imino sugar ring.

More recently, another related class of nucleoside phosphorylase and nucleosidase inhibitor compounds (known as the DAD-Me-Immucillins) has been developed. The location of the nitrogen atom in the imino sugar ring of this class of compounds is varied and/or the imino sugar moiety is linked to the nucleoside base analogue via a methylene bridge. The DAD-Me-Immucillins are described in U.S. Ser. No. 10/524,995.

Some of the Immucillins have also been identified as potent inhibitors of MTAP and MTAN. These are the subject of U.S. Pat. No. 7,098,334.

MTAP and MTAN function in the polyamine biosynthesis pathway, in purine salvage in mammals, and in the quorum sensing pathways in bacteria. MTAP catalyses the reversible phosphorolysis of MTA to adenine and 5-methylthio-α-D-ribose-1-phosphate (MTR-1P). MTAN catalyses the reversible hydrolysis of MTA to adenine and 5-methylthio-α-D-ribose, and of S-adenosyl-L-homocysteine (SAH), to adenine and S-ribosyl-homocysteine (SRH). The adenine formed is subsequently recycled and converted into nucleotides. Essentially, the only source of free adenine in the human cell is a result of the action of these enzymes. The MTR-1P is subsequently converted into methionine by successive enzymatic actions.

MTA is a by-product of the reaction involving the transfer of an aminopropyl group from decarboxylated S-adenosylmethionine to putrescine during the formation of spermidine. The reaction is catalyzed by spermidine synthase. Likewise, spermine synthase catalyses the conversion of spermidine to spermine, with concomitant production of MTA as a by-product. The spermidine synthase is very sensitive to product inhibition by accumulation of MTA. Therefore, inhibition of MTAP or MTAN severely limits the polyamine biosynthesis and the salvage pathway for adenine in the cells.

Likewise, MTA is the by-product of the bacterial synthesis of acylated homoserine lactones from S-adenosylmethionine (SAM) and acyl-acyl carrier proteins in which the subsequent lactonization causes release of MTA and the acylated homoserine lactone. The acylated homoserine lactone is a bacterial quorum sensing molecule in bacteria that is involved in bacterial virulence against human tissues. The homoserine lactone pathway will suffer feedback inhibition by the accumulation of MTA.

MTAP deficiency due to a genetic deletion has been reported with many malignancies. The loss of MTAP enzyme function in these cells is known to be due to homozygous deletions on chromosome 9 of the closely linked MTAP and p16/MTS1 tumour suppressor gene. As absence of p16/MTS1 is probably responsible for the tumour, the lack of MTAP activity is a consequence of the genetic deletion and is not causative for the cancer. However, the absence of MTAP alters the purine metabolism in these cells so that they are mainly dependent on the de novo pathway for their supply of purines.

MTA has been shown to induce apoptosis in dividing cancer cells, but to have the opposite, anti-apoptotic effect on dividing normal cells such as hepatocytes (E. Ansorena et al., Hepatology, 2002, 35: 274-280). Administration of MTA in circumstances where its degradation by MTAP is inhibited by an MTAP inhibitor will lead to greater circulatory and tissue levels of MTA and consequently an enhanced effect in the treatment of cancer.

MTAP and MTAN inhibitors may therefore be used in the treatment of diseases such as cancer, bacterial infections or protozoal parasitic infections, where it is desirable to inhibit MTAP or MTAN. Such treatments are described in U.S. Pat. No. 7,098,334 and U.S. Ser. No. 10/524,995.

The Immucillins and DAD-Me-Immucillins are also useful as inhibitors of nucleoside hydrolases. These enzymes catalyse the hydrolysis of nucleosides. They are not found in mammals, but are required for nucleoside salvage in some protozoan parasites. Certain protozoan parasites use nucleoside phosphorylases instead of or as well as nucleoside hydrolases for this purpose. Inhibitors of nucleoside hydrolases and phosphorylases can be expected to interfere with the metabolism of the parasite and therefore be usefully employed against protozoan parasites.

The Immucillins and the DAD-Me-Immucillins therefore represent two classes of compounds which are potent inhibitors of PNP, MTAP, MTAN and/or nucleoside hydrolases. Initially, work in this area of drug design focused on the synthesis of these compounds in their natural enantiomeric forms. Thus, to date, all of the active inhibitor compounds have incorporated the D-enantiomeric form of the imino sugar moiety. It was thought that the D-form of the sugar was necessary in order for the compounds to exhibit the requisite inhibitory activity.

The X-ray crystal structure of one of the inhibitor compounds (DAD-Me-Immucillin-H) bound to Mycobacterium tuberculosis PNP has been described (A. Lewandowicz, W. Shi, G. B. Evans, P. C. Tyler, R. H. Furneaux, L. A. Basso, D. S. Santos, S. C. Almo and V. L. Schramm, Biochemistry, 42 (2003) 6057-6066.). The complex of this inhibitor with PNP has favourable hydrogen bonds to almost every hydrogen bond donor-acceptor site in the complex. Even a slight structural change can disrupt this favourable hydrogen bonding pattern, as demonstrated by energetic mapping of transition state analogue interactions with human and Plasmodium falciparum PNPs (A. Lewandowicz, E. A. T. Ringia, L.-M. Ting, K. Kim, P. C. Tyler, G. B. Evans, O. V. Zubkova, S. Mee, G. F. Painter, D. H. Lenz, R. H. Furneaux and V. L. Schramm, J. Biol. Chem., 280 (2005) 30320-30328).

All indications have suggested that the D-form of the imino sugar is the preferable form for designing and synthesising suitable inhibitor compounds. Not only does the D-form correspond to the naturally occurring sugar form, but it has been demonstrated that the binding of the inhibitors is acutely sensitive to structural modifications.

However, despite all the evidence pointing to the D-enantiomeric forms as being the potent inhibitors, the applicants have now surprisingly found that the L-enantiomeric forms of the DAD-Me-Immucillins are also potent inhibitors of PNP MTAP, MTAN, and/or nucleoside hydrolases.

It is therefore an object of the present invention to provide novel inhibitors of PNP, MTAP, MTAN, and/or nucleoside hydrolases, or to at least provide a useful choice.

### STATEMENTS OF INVENTION

In a first aspect the invention provides a compound of formula (I):

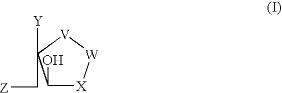

wherein:
   V is selected from CH₂ and NH, and W is selected from NR¹ and NR²; or V is selected from NR¹ and NR², and W is selected from CH₂ and NH;
   X is selected from CH₂ and CHOH in the R or S-configuration;
   Y is selected from hydrogen, halogen and hydroxy, except where V is selected from NH, NR¹ and NR² then Y is hydrogen;
   Z is selected from hydrogen, halogen, hydroxy, SQ, OQ and Q, where Q is an optionally substituted alkyl, aralkyl or aryl group;
   R¹ is a radical of the formula (II)

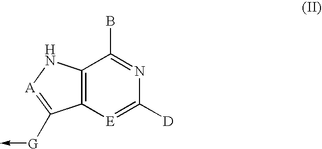

R² is a radical of the formula (III)

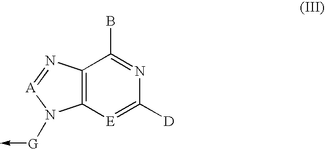

A is selected from N, CH and CR, where R is selected from halogen, optionally substituted alkyl, aralkyl or aryl, OH, NH₂, NHR³, NR³R⁴ and SR⁵, where R³, R⁴ and R⁵ are each optionally substituted alkyl, aralkyl or aryl groups;
B is selected from OH, NH₂, NHR⁶, SH, hydrogen and halogen, where R⁶ is an optionally substituted alkyl, aralkyl or aryl group;
D is selected from OH, NH₂, NHR⁷, hydrogen, halogen and SCH₃, where R⁷ is an optionally substituted alkyl, aralkyl or aryl group;
E is selected from N and CH;
G is selected from CH₂ and NH, or G is absent, provided that where W is NR¹ or NR² and G is NH then V is CH₂, and provided that where V is NR¹ or NR² and G is NH then W is CH₂;
or a tautomer thereof, or a pharmaceutically acceptable salt thereof, or an ester thereof, or a prodrug thereof.

Preferably Z is selected from hydrogen, halogen, hydroxy, SQ and OQ. More preferably Z is OH. Alternatively it is preferred that Z is SQ. In another preferred embodiment, Z is Q.

It is also preferred that V is CH₂. It is further preferred that X is CH₂. Additionally, it is preferred that G is CH₂.

Preferably W is NR¹. Alternatively it is preferred that W is NR². It is also preferred that where W is selected from NH, NR¹ or NR² then X is CH₂.

Preferred compounds of the invention include those where V, X and G are all CH₂, Z is OH and W is NR¹.

Other preferred compounds of the invention include those where V, X and G are all CH₂, Z is SQ and W is NR¹.

Preferably Y is hydrogen. Alternatively it is preferred that Y is hydroxy.

Preferably B is hydroxy. Alternatively it is preferred that B is NH₂.

Preferably A is CH. Alternatively it is preferred that A is N.

Preferably D is H. Alternatively it is preferred that D is NH₂.

It is also preferred that E is N.

It is preferred that any halogen is selected from chlorine and fluorine.

Q may be substituted with one or more substituents selected from OH, halogen (particularly fluorine or chlorine), methoxy, amino, or carboxy.

R³, R⁴, R⁵, R⁶ and R⁷ may each optionally be substituted with one or more substituents selected from OH or halogen, especially fluorine or chlorine.

Preferred compounds of the invention include:
(3S,4S)-1-[(9-deazaadenin-9-yl)methyl]-3-hydroxy-4-(hydroxymethyl)-pyrrolidine;
(3S,4S)-1-[(9-deazaadenin-9-yl)methyl]-3-hydroxy-4-methyl-pyrrolidine;
(3S,4S)-1-[(9-deazaadenin-9-yl)methyl]-3-hydroxy-4-(2-phenylethyl)-pyrrolidine;
(3S,4R)-1-[(9-deazaadenin-9-yl)methyl]-3-hydroxy-4-(methylthiomethyl)-pyrrolidine;
(3S,4R)-1-[(9-deazaadenin-9-yl)methyl]-3-hydroxy-4-(ethylthiomethyl)-pyrrolidine;
(3S,4R)-1-[(9-deazaadenin-9-yl)methyl]-3-hydroxy-4-(2-fluoroethylthiomethyl)-pyrrolidine;
(3S,4R)-1-[(9-deazaadenin-9-yl)methyl]-3-hydroxy-4-(2-hydroxyethylthiomethyl)-pyrrolidine;
(3S,4R)-1-[(9-deazaadenin-9-yl)methyl]-3-hydroxy-4-(propylthiomethyl)-pyrrolidine;
(3S,4R)-1-[(9-deazaadenin-9-yl)methyl]-3-hydroxy-4-(isopropylthiomethyl)-pyrrolidine;
(3S,4R)-1-[(9-deazaadenin-9-yl)methyl]-3-hydroxy-4-(butylthiomethyl)-pyrrolidine;
(3S,4R)-1-[(9-deazaadenin-9-yl)methyl]-3-hydroxy-4-(cyclohexylylthiomethyl)-pyrrolidine;
(3S,4R)-1-[(9-deazaadenin-9-yl)methyl]-3-hydroxy-4-(cyclohexylmethylthiomethyl)-pyrrolidine;
(3S,4R)-1-[(9-deazaadenin-9-yl)methyl]-3-hydroxy-4-(cyclopentylthiomethyl)-pyrrolidine;
(3S,4R)-1-[(9-deazaadenin-9-yl)methyl]-3-hydroxy-4-(phenylthiomethyl)-pyrrolidine;
(3S,4R)-1-[(9-deazaadenin-9-yl)methyl]-3-hydroxy-4-(4-fluorophenylthiomethyl)-pyrrolidine;
(3S,4R)-1-[(9-deazaadenin-9-yl)methyl]-3-hydroxy-4-(4-chlorophenylthiomethyl)-pyrrolidine;
(3S,4R)-1-[(9-deazaadenin-9-yl)methyl]-3-hydroxy-4-(3-chlorophenylthiomethyl)-pyrrolidine;
(3S,4R)-1-[(9-deazaadenin-9-yl)methyl]-3-hydroxy-4-(4-methylphenylthiomethyl)-pyrrolidine;
(3S,4R)-1-[(9-deazaadenin-9-yl)methyl]-3-hydroxy-4-(3-methylphenylthiomethyl)-pyrrolidine;
(3S,4R)-1-[(9-deazaadenin-9-yl)methyl]-3-hydroxy-4-(benzylthiomethyl)-pyrrolidine;
(3S,4S)-1-[(9-deazaadenin-9-yl)methyl]-3-acetoxy-4-(acetoxymethyl)-pyrrolidine.
(3S,4S)-1-[(9-deazaguanin-9-yl)methyl]-3-hydroxy-4-(hydroxymethyl)-pyrrolidine;
(3S,4R)-1-[(9-deazaguanin-9-yl)methyl]-3-hydroxy-4-(methylthiomethyl)-pyrrolidine;
(3S,4S)-1-[(9-deazaguanin-9-yl)methyl]-3-hydroxy-4-methyl-pyrrolidine;
(3S,4S)-1-[(9-deazahypoxanthin-9-yl)methyl]-3-hydroxy-4-(hydroxymethyl)-pyrrolidine;
(3S,4R)-1-[(9-deazahypoxanthin-9-yl)methyl]-3-hydroxy-4-(methylthiomethyl)-pyrrolidine;
(3S,4S)-1-[(9-deazahypoxanthin-9-yl)methyl]-3-hydroxy-4-methyl-pyrrolidine;
(3S,4S)-1-[(9-deaza-8-fluoro-hypoxanthin-9-yl)methyl]-3-hydroxy-4-(hydroxymethyl)-pyrrolidine;
(3R,4R)-1-[(9-deazahypoxanthin-9-yl)methyl]-3,4-dihydroxy-4-(hydroxymethyl)-pyrrolidine;
(3R,4S)-1-[(9-deazahypoxanthin-9-yl)methyl]-3,4-dihydroxy-4-(methylthiomethyl)-pyrrolidine;
(3S,4S)-1-[(9-deazaxanthin-9-yl)methyl]-3-hydroxy-4-(hydroxymethyl)-pyrrolidine;
(3S,4S)-1-[(9-deazaxanthin-9-yl)methyl]-3-hydroxy-4-(methylthiomethyl)-pyrrolidine;
(3S,4S)-1-[(6-chloro-9-deazapurin-9-yl)methyl]-3-hydroxy-4-(hydroxymethyl)-pyrrolidine;
(3S,4S)-1-[(6-azido-9-deazapurin-9-yl)methyl]-3-hydroxy-4-(hydroxymethyl)-pyrrolidine; and
(3S,4S)-1-[(8-aza-9-deazaadenin-9-yl)methyl]-3-hydroxy-4-(hydroxymethyl)-pyrrolidine;
(3S,4S)-1-[(8-aza-9-deazaadenin-9-yl)methyl]-3-hydroxy-4-methyl-pyrrolidine;
(3S,4R)-1-[(8-aza-9-deazaadenin-9-yl)methyl]-3-hydroxy-4-(benzylthiomethyl)-pyrrolidine;
(3S,4R)-1-[(8-aza-9-deazaadenin-9-yl)methyl]-3-hydroxy-4-(methylthiomethyl)-pyrrolidine;
(3S,4R)-1-[(8-aza-9-deazaadenin-9-yl)methyl]-3-hydroxy-4-(ethylthiomethyl)-pyrrolidine;
(3S,4R)-1-[(8-aza-9-deazaadenin-9-yl)methyl]-3-hydroxy-4-(propylthiomethyl)-pyrrolidine;
(3S,4R)-1-[(8-aza-9-deazaadenin-9-yl)methyl]-3-hydroxy-4-(isopropylthiomethyl)-pyrrolidine;
(3S,4R)-1-[(8-aza-9-deazaadenin-9-yl)methyl]-3-hydroxy-4-(butylthiomethyl)-pyrrolidine;
(3S,4R)-1-[(8-aza-9-deazaadenin-9-yl)methyl]-3-hydroxy-4-(phenylthiomethyl)-pyrrolidine;
(3S,4R)-1-[(8-aza-9-deazaadenin-9-yl)methyl]-3-hydroxy-4-(4-fluorophenylthiomethyl)-pyrrolidine;
(3S,4R)-1-[(8-aza-9-deazaadenin-9-yl)methyl]-3-hydroxy-4-(4-chlorophenylthiomethyl)-pyrrolidine;
(3S,4R)-1-[(8-aza-9-deazaadenin-9-yl)methyl]-3-hydroxy-4-(3-chlorophenylthiomethyl)-pyrrolidine;
(3S,4R)-1-[(8-aza-9-deazaadenin-9-yl)methyl]-3-hydroxy-4-(4-methylphenylthiomethyl)-pyrrolidine;
(3S,4R)-1-[(8-aza-9-deazaadenin-9-yl)methyl]-3-hydroxy-4-(3-methylphenylthiomethyl)-pyrrolidine;
(3S,4S)-1-[(8-aza-9-deazaguanin-9-yl)methyl]-3-hydroxy-4-(hydroxymethyl)-pyrrolidine;
(3S,4S)-1-[(8-aza-9-deazaguanin-9-yl)methyl]-3-hydroxy-4-methyl-pyrrolidine;
(3S,4S)-1-[(8-aza-9-deazaguanin-9-yl)methyl]-3-hydroxy-4-(methylthiomethyl)-pyrrolidine;
(3S,4S)-1-[(8-aza-9-deazahypoxanthin-9-yl)methyl]-3-hydroxy-4-(hydroxymethyl)-pyrrolidine;
(3S,4S)-1-[(8-aza-9-deazahypoxanthin-9-yl)methyl]-3-hydroxy-4-methyl-pyrrolidine;
(3S,4S)-1-[(8-aza-9-deazahypoxanthin-9-yl)methyl]-3-hydroxy-4-(methylthiomethyl)-pyrrolidine;
(3S,4S)-1-[(8-aza-9-deazaxanthin-9-yl)methyl]-3-hydroxy-4-(hydroxymethyl)-pyrrolidine; and
(3S,4S)-1-[(8-aza-9-deazaxanthin-9-yl)methyl]-3-hydroxy-4-(methylthiomethyl)-pyrrolidine.

According to another aspect of the invention, there is provided a pharmaceutical composition comprising a pharmaceutically effective amount of a compound of the formula (I).

Preferably the pharmaceutical composition comprises one of the above preferred compounds of the invention.

In another aspect of the invention there is provided a method of treating or preventing diseases or conditions in which it is desirable to inhibit PNP comprising administering a pharmaceutically effective amount of a compound of formula (I) to a patient requiring treatment. The diseases or conditions include cancer, bacterial and parasitic infections, and T-cell mediated diseases such as psoriasis, lupus, arthritis and other autoimmune diseases. This aspect of the invention also includes use of the compounds for immunosuppression for organ transplantation. Preferably the compound is one of the above preferred compounds of the invention.

The parasitic infections include those caused by protozoan parasites such as those of the genera Giardia, Trichomonas, Leishmania, Trypanosoma, Crithidia, Herpetomonas, Leptomonas, Histomonas, Eimeria, Isopora and Plasmodium. The method can be advantageously applied with any parasite containing one or more nucleoside hydrolases inhibited by a compound of the invention when administered in an amount providing an effective concentration of the compound at the location of the enzyme.

In another aspect, the invention provides a method of treating or preventing diseases or conditions in which it is desirable to inhibit MTAP comprising administering a pharmaceutically effective amount of a compound of formula (I) to a patient requiring treatment. The diseases include cancer, for example prostate and head and neck tumours.

In another aspect, the invention provides a method of treating or preventing diseases or conditions in which it is desirable to inhibit MTAN comprising administering a pharmaceutically effective amount of a compound of formula (I) to a patient requiring treatment. The diseases include bacterial infections.

In another aspect the invention provides the use of a compound of formula (I) for the manufacture of a medicament for treating one or more of these diseases or conditions.

In still a further aspect of the invention there is provided a method of preparing a compound of formula (I).

In still a further aspect of the invention there is provided an intermediate useful in the preparation of a compound of formula (I).

### DETAILED DESCRIPTION

### Definitions

The term “alkyl” is intended to include both straight- and branched-chain alkyl groups. The same terminology applies to the non-aromatic moiety of an aralkyl radical. Examples of alkyl groups include: methyl group, ethyl group, n-propyl group, iso-propyl group, n-butyl group, iso-butyl group, sec-butyl group, t-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group and 1-methyl-2-ethylpropyl group.

The term “aryl” means an aromatic radical having 6 to 18 carbon atoms and includes heteroaromatic radicals. Examples include monocyclic groups, as well as fused groups such as bicyclic groups and tricyclic groups. Some examples include phenyl group, indenyl group, 1-naphthyl group, 2-naphthyl group, azulenyl, group, heptalenyl group, biphenyl group, indacenyl group, acenaphthyl group, fluorenyl group, phenalenyl group, phenanthrenyl group, anthracenyl group, cyclopentacyclooctenyl group, and benzocyclooctenyl group, pyridyl group, pyrrolyl group, pyridazinyl group, pyrimidinyl group, pyrazinyl group, triazolyl group, tetrazolyl group, benzotriazolyl group, pyrazolyl group, imidazolyl group, benzimidazolyl group, indolyl group, isoindolyl group, indolizinyl group, purinyl group, indazolyl group, furyl group, pyranyl group, benzofuryl group, isobenzofuryl group, thienyl group, thiazolyl group, isothiazolyl group, benzothiazolyl group, oxazolyl group, and isoxazolyl group.

The term “halogen” includes fluorine, chlorine, bromine and iodine.

The compounds are useful for the treatment of certain diseases and disorders in humans and other animals. Thus, the term “patient” as used herein includes both human and other animal patients.

The term “prodrug” as used herein means a pharmacologically acceptable derivative of the compound of formula (I) or formula (II), such that an in vivo biotransformation of the derivative gives the compound as defined in formula (I) or formula (II). Prodrugs of compounds of formula (I) or formula (II) may be prepared by modifying functional groups present in the compounds in such a way that the modifications are cleaved in vivo to give the parent compound.

The term “pharmaceutically acceptable salts” is intended to apply to non-toxic salts derived from inorganic or organic acids, including, for example, the following acid salts: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, p-toluenesulfonate, salicylate, succinate, sulfate, tartrate, thiocyanate, and undecanoate.

As used herein, the term “sulfonate leaving group” means an alkyl or aryl sulfonate such as methanesulfonate or benzenesulfonate, or a substituted form thereof such as bromobenzenesulfonate, trifluoromethanesulfonate or p-toluenesulfonate.

As used herein, the term “protecting group” means a group that selectively protects an organic functional group, temporarily masking the chemistry of that functional group and allowing other sites in the molecule to be manipulated without affecting the functional group. Suitable protecting groups are known to those skilled in the art and are described, for example, in Protective Groups in Organic Synthesis (3^{rd} Ed.), T. W. Greene and P. G. M. Wuts, John Wiley & Sons Inc (1999).

### Description of the Inhibitor Compounds

It is well known that chiral components of natural products occur predominantly in one of their enantiomeric forms. For sugars, these are the L- and D-modifications. Since enzymes work together with their substrates like a lock and key, one enantiomer, typically the naturally occurring species, is usually a better “fit” than the other. In the case of sugars, the D-form is naturally occurring, so work in the area of synthetic drug design is usually restricted to the investigation of D-sugars.

It is therefore surprising and unexpected that the compounds of the invention are inhibitors of PNP, MTAP, MTAN and/or nucleoside hydrolases, as the imino sugar moiety in these compounds is the L-enantiomeric form. It was previously thought that the D-enantiomer, being the naturally occurring form, would preferable for designing and synthesising suitable inhibitor compounds. In addition, it has been demonstrated that the D-enantiomers bind to the PNP enzyme with a number of favourable hydrogen bond contacts.

The compounds of the invention therefore represent a new class of inhibitors of PNP, MTAP, MTAN, and/or nucleoside hydrolases. As such, they are useful in treating diseases and conditions such as cancer, bacterial infections, parasitic infections, T-cell mediated diseases and other autoimmune diseases, and for immunosuppression for organ transplantation. Cancer means any type of cancer, including, but not limited to, cancers of the head, neck, bladder, bowel, skin, brain, CNS, breast, cervix, kidney, larynx, liver, esophagus, ovaries, pancreas, prostate, lung, stomach, testes, thyroid, uterus, as well as melanoma, leukaemia, lymphoma, osteosarcoma, Hodgkin's disease, glioma, sarcoma and colorectal, endocrine, gastrointestinal cancers.

### General Aspects

The compounds of the invention are useful in both free base form and in the form of salts.

It will be appreciated that the representation of a compound of formula (I), where B and/or D is a hydroxy group, is of the enol-type tautomeric form of a corresponding amide, and this will largely exist in the amide form. The use of the enol-type tautomeric representation is simply to allow fewer structural formulae to represent the compounds of the invention.

Similarly, it will be appreciated that the representation of a compound of formula (I), where B is a thiol group, is of the thioenol-type tautomeric form of a corresponding thioamide, and this will largely exist in the thioamide form. The use of the thioenol-type tautomeric representation is simply to allow fewer structural formulae to represent the compounds of the invention.

The active compounds may be administered to a patient by a variety of routes, including orally, parenterally, by inhalation spray, topically, rectally, nasally, buccally or via an implanted reservoir. The amount of compound to be administered will vary widely according to the nature of the patient and the nature and extent of the disorder to be treated. Typically the dosage for an adult human will be in the range less than 1 to 1000 milligrams, preferably 0.1 to 100 milligrams. The specific dosage required for any particular patient will depend upon a variety of factors, including the patient's age, body weight, general health, sex, etc.

For oral administration the compounds can be formulated into solid or liquid preparations, for example tablets, capsules, powders, solutions, suspensions and dispersions. Such preparations are well known in the art as are other oral dosage regimes not listed here. In the tablet form the compounds may be tableted with conventional tablet bases such as lactose, sucrose and corn starch, together with a binder, a disintegration agent and a lubricant. The binder may be, for example, corn starch or gelatin, the disintegrating agent may be potato starch or alginic acid, and the lubricant may be magnesium stearate. For oral administration in the form of capsules, diluents such as lactose and dried cornstarch may be employed. Other components such as colourings, sweeteners or flavourings may be added.

When aqueous suspensions are required for oral use, the active ingredient may be combined with carriers such as water and ethanol, and emulsifying agents, suspending agents and/or surfactants may be used. Colourings, sweeteners or flavourings may also be added.

The compounds may also be administered by injection in a physiologically acceptable diluent such as water or saline. The diluent may comprise one or more other ingredients such as ethanol, propylene glycol, an oil or a pharmaceutically acceptable surfactant.

The compounds may also be administered topically. Carriers for topical administration of the compounds of include mineral oil, liquid petrolatum, white petrolatum, propylene glycol, polyoxyethylene, polyoxypropylene compound, emulsifying wax and water. The compounds may be present as ingredients in lotions or creams, for topical administration to skin or mucous membranes. Such creams may contain the active compounds suspended or dissolved in one or more pharmaceutically acceptable carriers. Suitable carriers include mineral oil, sorbitan monostearate, polysorbate 60, cetyl ester wax, cetearyl alcohol, 2-octyldodecanol, benzyl alcohol and water.

The compounds may further be administered by means of sustained release systems. For example, they may be incorporated into a slowly dissolving tablet or capsule.

### Synthesis of the Inhibitor Compounds

As the skilled person will realise, the compounds of the invention may be synthesised using similar methods to those used for the synthesis of their D enantiomers.

One suitable synthetic procedure involves using a Mannich reaction to couple a 9-deazapurine or an 8-aza-9-deazapurine moiety (or their 2-aza-analogues) to a cyclic secondary amine.

In other words, a compound of the formula (I′)

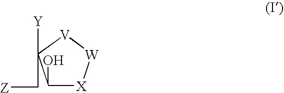

wherein: V is selected from CH₂ and NH, and W is NR¹; or
V is NR¹, and W is selected from CH₂ and NH;
X is selected from CH₂ and CHOH in the R or S-configuration, except where W is selected from NH and NR¹, then X is CH₂;
Y is selected from hydrogen, halogen and hydroxy, except where V is selected from NH and NR¹, then Y is hydrogen;
Z is selected from hydrogen, halogen, hydroxy, a sulfonate leaving group, SQ, OQ and Q, where Q is an optionally substituted alkyl, aralkyl or aryl group; and
R¹ is a radical of the formula (II′)

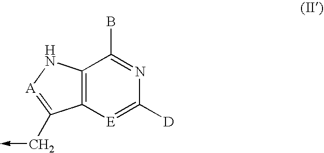

wherein:
A is selected from N, CH and CR², where R² is selected from halogen, optionally substituted alkyl, aralkyl or aryl, OH, NH₂, NHR³, NR³R⁴ and SR⁵, where R³, R⁴ and R⁵ are each optionally substituted alkyl, aralkyl or aryl groups;
B is selected from OH, NH₂, NHR⁶, SH, hydrogen and halogen, where R⁶ is an optionally substituted alkyl, aralkyl or aryl group;
D is selected from OH, NH₂, NHR⁷, hydrogen, halogen and SCH₃, where R⁷ is an optionally substituted alkyl, aralkyl or aryl group; and
E is selected from N and CH;
   may be prepared by reaction of a compound of the formula (IV)

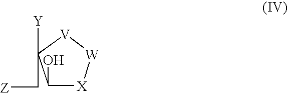

wherein:
V is selected from CH₂ and NH, and W is NH; or
V is NH, and W is selected from CH₂ and NH;
X is selected from CH₂ and CHOH in the R or S-configuration, except where W is NH, then X is CH₂;
Y is selected from hydrogen, halogen and hydroxy, except where V is selected from NH, then Y is hydrogen; and
Z is selected from hydrogen, halogen, hydroxy, a sulfonate leaving group, SQ, OQ and Q, where Q is an optionally substituted alkyl, aralkyl or aryl group;
   with a compound of the formula (V)

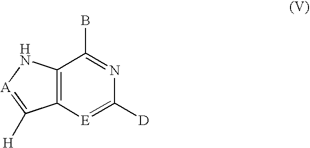

wherein A, B, D, and E are as defined above;
   and with formaldehyde or a formaldehyde equivalent.

Compounds of the formula (IV) as defined above may be prepared by known methods, as described in WO 2004/018496 and the references cited therein.

Compounds of formula (V) defined above may be prepared by known methods. In particular, processes for the preparation of the compounds 3H,5H-pyrrolo[3,2-d]pyrimidin-4-one (9-deazahypoxanthine) and 2-amino-3H,5H-pyrrolo[3,2-d]pyrimidin-4-one (9-deazaguanine), compounds A and B shown below, are described in U.S. Pat. No. 6,693,193 and in R. H. Furneaux and P. C. Tyler, J. Org. Chem., 64 (1999) 8411-8412. Further, 9-deazaadenine (C) can be prepared by treatment of 9-deazahypoxanthine (A) with POCl₃ and then with ethanolic ammonia.

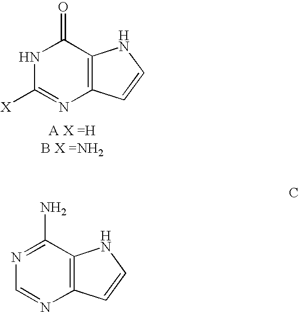

One advantage of the Mannich process is that neither the amine nor the heterocyclic component needs to have protecting groups on the functional groups that are not directly involved in the reaction chemistry. Nevertheless, there may be occasions where it is advantageous to utilize a protected form of a compound of formula (IV) and/or formula (V) as components in the reaction.

Suitably protected forms of compounds of formula (IV) are described in U.S. Pat. No. 5,985,848, U.S. Pat. No. 6,066,722, and U.S. Pat. No. 7,109,331. It is essential that suitably protected forms of compounds of the formula (V) have a proton at position-9 of the 9-deazapurine or 8-aza-9-deazapurine moiety (or their 2-aza-analogues).

Suitably protected forms of compounds of formula (V) are described in U.S. Ser. No. 10/524,995. It is essential that protected forms of compounds of the formula (IV) have an unprotected ring amino group.

### EXAMPLES

The following examples further illustrate the invention. It is to be appreciated that the invention is not limited to the examples.

### General

All reagents were used as supplied; anhydrous solvents were obtained commercially. Air sensitive reactions were carried out under argon. Organic solutions were dried over MgSO₄ and the solvents were evaporated under reduced pressure. Chromatography solvents were distilled prior to use. Thin layer chromatography (t.l.c.) was performed on glass or aluminium sheets coated with 60 F₂₅₄ silica. Organic compounds were visualised under uv light or by use of a spray or dip of cerium(IV) sulfate (0.2%, w/v) and ammonium molybdate (5%) in sulfuric acid (2M), one of I₂ (0.2%) and KI (7%) in H₂SO₄ (M) or, for nitrogen-containing compounds, p-(N,N-dimethylamino)benzaldehyde (1%) in HCl (37%)-MeOH, 1:3 (100 ml) (Erlich reagent). Flash column chromatography was performed on Scharlau silica gel 60 (40-60 μm). Melting points were recorded on a Reichert hot stage microscope and are uncorrected. Optical rotations were recorded on a Perkin-Elmer 241 polarimeter with a path length of 1 dm and are in units of 10⁻¹ deg cm²g⁻¹; concentrations are in g/100 ml.

NMR spectra were recorded on a Bruker AC300E spectrometer. ¹H spectra at 300 MHz were measured in CDCl₃ or CD₃OD (internal reference Me₄Si, δ 0), and ¹³C spectra at 75.5 MHz in CDCl₃ (reference, solvent centre line, δ 77.0) or CD₃OD (reference, solvent centre line δ 49.0). Assignments of ¹H and ¹³C resonances were based on 2D (¹H-¹H DQF-COSY, ¹H-¹³C HSQC) spectra, and DEPT experiments gave unambiguous data on the numbers of protons bonded to each carbon atom. The assignments of the ¹³C resonances were consistent with the multiplicities observed. Coupling constants (J) are quoted in Hz. Positive ion fast atom bombardment (FAB+) HRMS were measured on a VG 7070 instrument in a glycerol matrix, and positive ion electron impact (EI+) HRMS were measured on a VG 70SE instrument. Microanalyses were carried out by the Campbell Microanalytical Laboratory, University of Otago.

### 1. (3S,4S)-4-(Hydroxymethyl)pyrrolidin-3-ol and its hydrochloride

The synthesis of this compound was carried out as described previously (WO 2005/033076).

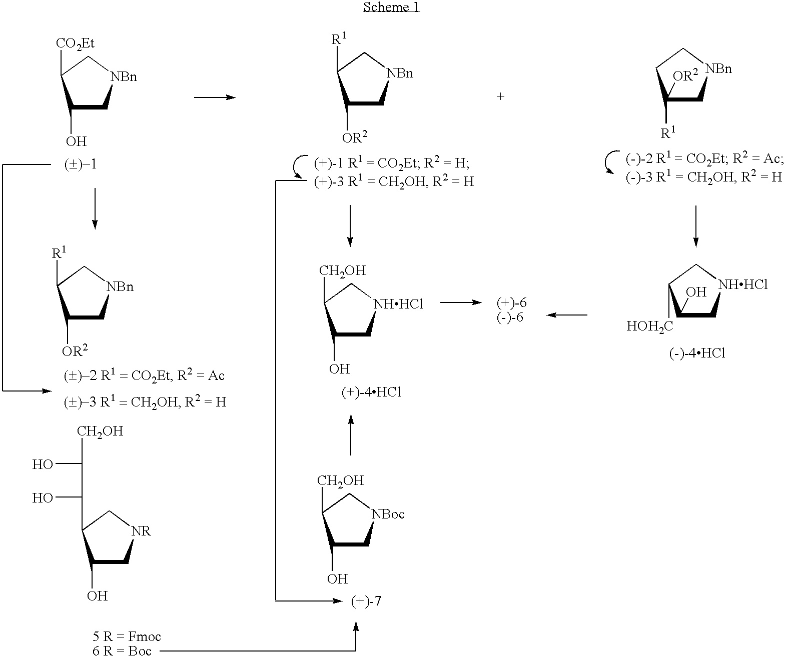

### Ethyl (R,S/S,R)-1-benzyl-4-hydroxypyrrolidine-3-carboxylate [(±)-1]

This compound was prepared as previously described (E. Jaeger and J. H. Biel, J. Org. Chem., 1965, 30, 740-744) but ethyl N-benzyl-N-(2-carbethoxyethyl)glycinate, as prepared by the method of Pinto et al. (A. C. Pinto, R. V. Abdala and P. R. R. Costa, Tetrahedron: Asymm., 2000, 11, 4239-4243) was used as well as the Dieckmann cyclization conditions described by Deshmukh et al. (M. N. Deshmukh, K. K. Gangakhedkar and U.S. Kumar, Synth. Commun., 1996, 26, 1657-1661). The racemic trans-isomer was purified by chromatography (EtOAc-hexanes, 1:2→1:1→EtOAc) and the resulting gum crystallized at −20° C. (44% from the glycinate on the 5 mmol scale). A small sample was recrystallized at −20° C. from EtOAc-hexanes to give colourless needles, mp 52-53° C., NMR δ_{H} (300 MHz; CDCl₃): 1.26 (3H, t, J 7.1, CH₂CH₃), 2.32 (1H, br. s, OH, exchanged to D₂O), 2.55 (1H, dd, J_{2,2′} 9.4, J_{2,3} 7.4, H-2), 2.65 (1H, dd, J_{5,5′} 10.0, J_{5,4} 5.5, H-5), 2.76 (1H, dd, J_{5′,4} 2.8, H-5′), 2.95 (1H, dt, J_{3,2}=J_{3,2′} 8.0, J_{3,4} 3.3, H-3), 3.12 (1H, t, J 9.0, H-2′), 3.64 (2H, s, PhCH₂), 4.16 (2H, q, J 7.1 CH₂CH₃), 4.51 (1H, m, H-4), 7.22-7.37 (5H, m, Ar); δ_{C} (75.5 MHz; CDCl₃) 14.2 (Me), 53.1 (C-3), 55.3 (C-2), 59.7 (PhCH₂), 60.8 (CH₃CH₂), 61.9 (C-5), 74.1 (C-4), 127.1 (ArH), 128.3 (ArH), 128.8 (ArH), 138.2 (Ar), 173.3 (CO); HRMS (EI+) m/z 249.1365; C₁₄H₁₉NO₃ (M⁺) requires 249.1365. (Found: C, 67.6; H, 7.5; N, 5.6; C₁₄H₁₉NO₃ requires C, 67.5; H, 7.7; N, 5.6%).

### Ethyl (R,S/S,R)-4-(acetyloxy)-1-benzylpyrrolidine-3-carboxylate [(±)-2]

Racemate 1 (100 mg, 0.4 mmol) was dissolved in a mixture of pyridine (4 ml) and Ac₂O (2 ml) and left at 20° C. overnight. The solvent was evaporated and the resulting oil dissolved in EtOAc and washed with aqueous NaHCO₃ (saturated), dried and the solvent was again evaporated. The residue was chromatographed (EtOAc-hexanes, 15:85) to afford diester (±)-2 as a colourless oil (111 mg, 95%) which was stored at −20° C., NMR δ_{H} (300 MHz; CDCl₃) 1.25 (3H, t, J 7.1, CH₂CH₃), 2.04 (3H, s, COCH₃), 2.50 (1H, t, J_{2,2′}=J_{2,3} 8.3, H-2), 2.74-2.87 (2H, m, H-5,5′), 3.06 (1H, dt, J_{3,2}=J_{3,2′} 7.9, J_{3,4} 3.9, H-3), 3.15 (1H, t, J 8.5, H-2′), 3.59 (1H, d, J 12.9, PhCHH), 3.65 (1H, d, PhCHH), 4.16 (2H, q, J 7.1, CH₂CH₃) 5.40 (1H, m, H-4), 7.22-7.38 (5H, m, Ar); δ_{C} (75.5 MHz; CDCl₃) 14.1 (CH₂CH₃), 21.0 (COCH₃), 50.1 (C-3), 56.0 (C-2), 59.5 (PhCH₂ or C-5), 59.6 (PhCH₂ or C-5), 61.0 (CH₂CH₃), 76.0 (C-4), 127.2 (ArH), 128.3 (ArH), 128.7 (ArH), 138.0 (Ar), 170.5 (CO), 172.3 (CO); HRMS (FAB+) m/z 292.1563; C₁₆H₂₂NO₄ (M+H)⁺ requires 292.1549.

### (R,R/S,S)-1-Benzyl-4-(hydroxymethyl)pyrrolidin-3-ol [(±)-3]

Racemate 1 (500 mg, 2.01 mmol) was dissolved in dry Et₂O-dry THF, (10 ml:5 ml) and cooled in an ice bath. Lithium aluminium hydride in Et₂O (4.2 ml, M, 4.2 mmol) was added, and the mixture warmed to 20° C. and stirred for 1 h. After cooling of the solution in an ice bath excess hydride was quenched by the dropwise addition of water (0.50 ml) and the mixture was extracted with EtOAc. The organic extract was washed with aqueous NaHCO₃ (saturated), dried and evaporated to give an oily residue that was chromatographed [CH₂Cl₂-MeOH—NH₄OH (0.88), 95:5:0.5→90:10:0.5] to give racemic diol 3 as a colourless gum (364 mg, 88%), NMR δ_{H} (300 MHz; CD₃OD) 2.18 (1H, m, H-4), 2.34 (1H, dd, J_{5,5′} 9.6, J_{5,4} 6.6, H-5), 2.55 (1H, dd, J_{2,2′} 10.0, J_{2,3} 4.1, H-2), 2.72 (1H, dd, J_{2′,3} 6.3, H-2′), 2.89 (1H, t, J_{5′,4}=J_{5′,5} 8.8, H-5′), 3.47-3.68 (4H, m, PhCH₂, CH₂O), 4.00 (1H, m, H-3), 7.20-7.42 (5H, m, Ar); δ_{C} (75.5 MHz; CD₃OD) 51.2 (C-4), 57.3 (C-5), 61.5 (PhCH₂ or CH₂O), 63.1 (C-2), 64.2 (PhCH₂ or CH₂O), 74.1 (C-3) 128.3 (ArH), 129.3 (ArH), 130.2 (ArH), 139.4 (Ar); HRMS (FAB+) m/z 208.1346; C₁₂H₁₈NO₂ (M+H)⁺ requires 208.1338.

### Ethyl (3S,4R)-1-benzyl-4-hydroxypyrrolidine-3-carboxylate [(±)-1] and ethyl (3R,4S)-4-(acetyloxy)-1-benzylpyrrolidine-3-carboxylate [(−)-2]

Vinyl acetate (6.66 ml, 72.21 mmol) and Novozyme® 435 lipase from Candida antarctica (4.2 g, Novozymes Australia Pty. Ltd, batch LC200207) were added sequentially to a solution of (±)-1 (6.00 g, 24.1 mmol) in tert-butyl methyl ether (200 ml). The mixture was stirred at 40° C. for 2.5 h, filtered through Celite®, the solids were washed with a little ethyl acetate and the combined filtrates were washed with aqueous NaHCO₃ (saturated), dried and evaporated. ¹H NMR analysis indicated that the residue consisted of alcohol 1 and acetate 2 in equimolar proportions. It was chromatographed (EtOAc-hexanes, 6:4) to give first (−)-2 as a colourless gum (3.44 g, 97%) that was stored at −20° C., [α]_{D}²¹−41.5 (c 0.74, CHCl₃). The ¹H NMR spectrum was identical to that for compound (±)-2 above. Further elution of the column with EtOAc gave (+)-1 also as a colourless gum which crystallized at −20° C. (2.53 g, 85%), mp 51-52° C., [α]_{D}²¹+16.9 (c 0.71, CHCl₃). The ¹H NMR spectrum was identical to that for compound (±)-1 above.

Repetition of the enzymic acetylation with (±)-1 (0.80 g, 3.21 mmol) under the same conditions, but for 100 min, gave a mixture of 1 and 2 in the approximate ratio of 1.2:1 (¹H NMR determination). After chromatographic separation, pure (−)-2 (406 mg, 96%), [α]_{D}²¹−41.8 (c 0.895, CHCl₃) and impure (+)−1 (0.393 g, 89%), [α]_{D}²¹+14.0 (c 0.81 CHCl₃) were isolated. The latter contained about 10% of the unreacted (−)-enantiomer.

### (3R,4R)-1-Benzyl-4-(hydroxymethyl)pyrrolidin-3-ol [(+)-3]

Compound (+)-1 (2.53 g, 10.15 mmol) was reduced, as indicated for the racemic compound, to give(+)-3 as a colourless gum (1.54 g, 73%), [α]_{D}²¹+33.0 (c 0.75, MeOH). The ¹H NMR spectrum was identical to that of compound (±)-3.

### tert-Butyl (3R,4R)-3-hydroxy-4-(hydroxymethyl)pyrrolidine-1-carboxylate [(+)-7]

A. From diol (+)-3. Pd—C (300 mg, 10%) was added to a stirred solution of the diol (+)-3 (1.49 g, 7.19 mmol) and di-tert-butyl dicarbonate (1.63 g, 7.47 mmol) in MeOH (30 ml), and hydrogen was added from a balloon over 24 h. The mixture was filtered through Celite®, the solvent was evaporated and the residue was chromatographed (EtOAc-MeOH, 19:1) to afford the N-Boc protected pyrrolidine (+)-7 as a colourless gum (1.56 g, 100%), [α]_{D}²¹+15.9 (c 1.09, MeOH), in good agreement with the value derived from the sample made by method B.

B. From 1,2:5,6-di-O-isopropylidine-α-D-glucose. 3-C-Azidomethyl-3-deoxy-1,2:5,6-di-O-isopropylidene-α-D-glucose (42.6 g, 142 mmol), which was made from 1,2:5,6-di-O-isopropylidine-α-D-glucose, hydrolysed and reduced as previously described (V. V. Filichev and E. B. Pedersen, Tetrahedron, 2001, 57, 9163-9168) gave the unprotected pyrrolidine from which, in MeOH, (500 ml), compound 6 was obtained by treatment with di-tert-butyl dicarbonate (40 g, 185 mmol) and Et₃N (25.7 ml, 185 mmol). The volatiles were removed and the residue was adsorbed on silica gel and chromatographed to give crude carbamate 6 (26.7 g, 68%). The product was dissolved in EtOH (500 ml), cooled in an ice bath and oxidised by the dropwise addition of NaIO₄ (47 g, 0.22 mol) in water (500 ml). After recooling of the products in an ice-bath the product was reduced with NaBH₄ (7.3 g, 0.19 mmol) added portion-wise. The mixture was warmed to room temperature, the solids were removed by filtration, the volatiles by evaporation and the residue was purified by chromatography (CHCl₃-MeOH, 9:1). Compound (+)-7 was obtained as a light yellow syrup (17 g, 81%) which gave ¹H and ¹³C NMR data in agreement with those of the sample made by method A and with literature data (G. B. Evans, R. H. Furneaux, A. Lewandowicz, V. L. Schramm and P. C. Tyler, J. Med. Chem. 2003, 46, 5271-5276). A sample of compound (+)-7 (50 mg), prepared in this way, in EtOAc was further purified by washing with water and then brine to give a colourless syrup (28 mg) after solvent evaporation, [α]_{D}²¹+16.2 (c 0.795, MeOH).

### (3R,4R)-4-(Hydroxymethyl)pyrrolidin-3-ol [(+)-8] and its Hydrochloride [(+)-8.HCl]

A. From carbamate 7. A sample of compound (+)-7 (28 mg) was dissolved in MeOH (2 ml) and HCl (37%, 1 ml) and after a few mins the solvent was evaporated to give (+)-8.HCl, [α]_{D}²¹+18.9 (c 0.92, MeOH). The ¹H NMR spectrum was identical to that of the sample made from diol (+)-3 (method B).

B. From (+)-3. Diol (+)-3 (52 mg, 0.25 mmol) was dissolved in MeOH, HCOOH (98%) (9:1, 8 ml) and Pd—C (10%, 80 mg) was added (T. W. Greene and P. G. M. Wuts, Protective Groups in Organic Synthesis, 3rd ed., John Wiley and Sons, New York, 1999, p. 79). The mixture was heated under reflux for 30 min, filtered through Celite® and the solvent evaporated. Chromatography [CH₂Cl₂-MeOH—NH₄OH (0.88)—H₂O, 4:3:0.5:0.5] gave the unprotected pyrrolidine as a colourless gum (16 mg, 55%) which darkened slowly on standing. The ¹H NMR spectrum (CD₃OD) was in agreement with literature spectral data (V. V. Filichev, M. Brandt and E. B. Pedersen, Carbohydr. Res., 2001, 333, 115-122). The product was dissolved in MeOH (2 ml), HCl (5%, 1 ml) and the solvents were evaporated to give the hydrochloride (+)-8.HCl (21 mg, 55%) as a colourless gum, [α]_{D}²¹+19.1 (c 1.05, MeOH), lit.²³ [α]_{D}²⁵+19.0 (c 1.0, MeOH). The ¹H NMR spectrum (D₂O) was in agreement with the literature spectral data (S. Karlsson and H.-E. Högberg, Tetrahedron: Asymmetry, 2001, 12, 1977-1982) and was identical to that of the compound made by method A.

### (3S,4S)-1-Benzyl-4-(hydroxymethyl)pyrrolidin-3-ol [(−)-3]

Compound (−)-2 (400 mg, 1.37 mmol) was dissolved in Et₂O (9 ml) and THF (4 ml) and treated with lithium aluminium hydride in Et₂O (5.62 ml, 1M, 5.62 mmol) as described for the preparation of compound (±)-3 above to afford (−)-3 as a colourless gum (190 mg, 67%), [α]_{D}²¹−33.4 (c 0.805, MeOH). The ¹H NMR spectrum was identical to that of (±)-3.

### (3S,4S)-4-(Hydroxymethyl)pyrrolidin-3-ol and its hydrochloride [(−)-8.HCl]

Compound (−)-3 (189 mg, 0.91 mmol) was de-N-benzylated (T. W. Greene and P. G. M. Wuts, Protective Groups in Organic Synthesis, 3rd ed., John Wiley and Sons, New York, 1999, p. 79) as for the (+)-enantiomer to give the unprotected amine as a colourless gum (107 mg, 100%), a portion of which (30 mg) was converted to the hydrochloride salt (−)-8.HCl (39 mg), [α]_{D}²¹−18.9 (c 0.74, MeOH), lit.²³ [α]_{D}²⁵−18.7 (c 1.2, MeOH). The ¹H NMR spectrum (D₂O) was in agreement with the literature data (S. Karlsson and H.-E. Högberg, Tetrahedron: Asymmetry, 2001, 12, 1977-1982) and was identical to that of (+)-8. HCl.

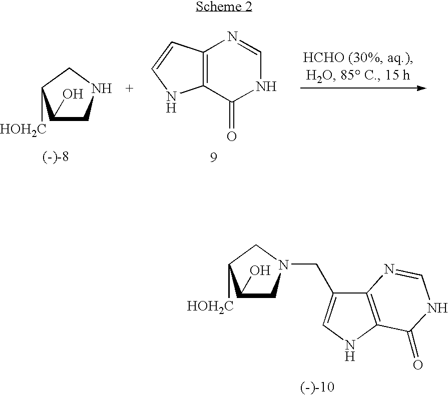

### (3S,4S)-1-[(9-Deazahypoxanthin-9-yl)methyl]-4-(hydroxymethyl)pyrrolidin-3-ol [L-DADMe-ImmH, (−)-10]

To a solution of (3S,4S)-4-(hydroxymethyl)pyrrolidin-3-ol free base (−)-8, (77 mg, 0.66 mmol) in H₂O (1.5 ml) were added 9-deazahypoxanthine (9) (R. H. Furneaux and P. C. Tyler, J. Org. Chem., 1999, 64, 8411-8412) (81 mg, 0.60 mmol) and aqueous formaldehyde (53 μl, 12.3M, 0.65 mmol). The mixture was heated at 85° C. for 15 h (a small amount of precipitate formed), silica gel was added to absorb the solvent, the solvent was evaporated and the granular residue added to a column of silica gel and eluted with CH₂Cl₂-MeOH—NH₄OH (0.88), 5:4.5:0.5 to afford the nucleoside analogue (−)-10 as a colourless solid (82 mg, 48%) after washing with a little cold MeOH, [α]_{D}²¹−16.8 (c 0.71, H₂O). A sample of the (3R,4R)-enantiomer (+)-10, prepared during the present work, and ultimately derived from D-glucose via the sequence (+)-6→(+)-7→(+)-8→(+)-10, had [α]_{D}²¹+16.9 (c 0.935, H₂O). The ¹H NMR spectrum of compound (−)-10 was in agreement with the literature data for (+)-10 (G. B. Evans, R. H. Furneaux, A. Lewandowicz, V. L. Schramm and P. C. Tyler, J. Med. Chem., 2003, 46, 5271-5276) and with the spectrum of the latter isomer made during the present work.

### Biological Data

Kinetic studies of the interactions between compounds (+)-10 and (−)-10 and human, plasmodial and bovine PNPases were carried out by the methods previously reported (R. W. Miles, P. C. Tyler, R. H. Furneaux, C. K. Bagdassarian, and V. L. Schramm, Biochemistry, 1998, 37, 8615-8621; G. B. Evans, R. H. Furneaux, A. Lewandowicz, V. L. Schramm and P. C. Tyler, J. Med. Chem. 2003, 46, 3412-3423) and the results are given in Table 1. The inhibition constants K_{I} are the dissociation constants for the enzyme-inhibitor complex measured from initial reaction rates. For many, but not all, immucillin inhibitors, a slow-onset of inhibition then occurs consequent upon a time dependent conformational change in the enzyme that leads to tighter binding characterised by the constant K_{I}* (J. F. Morrison and C. T. Walsh, Adv, Enzymol. Relat. Areas Mol. Biol., 1988, 61, 201-310).

To ensure that the inhibition observed with compound [(−)-10] was not due to small proportions of residual, more active, D-compound, a sample of the L-enantiomer was pre-treated with 0.5-1.0 molar equivalents of human PNPase and the product was subjected to ultrafiltration. In this way, [(+)-10] gave a sample that inhibited PNPases with kinetic parameters unchanged relative to those of the original preparation. Accordingly, based on the error limits of the kinetic constant for inhibition, it was concluded that no more than 2% of the D-enantiomer could have been present as a contaminant in the initial inhibitor (−)-10.

TABLE 1
Kinetic data for the inhibition of human, plasmodial
and bovine PNPases by the enantiomers of DADMe-ImmH
[(+)-10 and (−)-10]

| Compound | Enzyme source | Ki (nM) | Ki* (nM) |
|---|---|---|---|
| [(+)-10] | H. sapiens | 1.1 ± 0.1 | 0.016 ± 0.001 |
| | Plasmodium falciparum | 0.50 ± 0.04 | Not observed |
| | B. taurus | 2.1 ± 0.3 | 0.110 ± 0.014 |
| [(−)-10] | H. sapiens | 1.5 ± 0.1 | 0.68 ± 0.26 |
| | Plasmodium falciparum | 1700 ± 300 | 80 ± 7 |
| | B. taurus | 19 ± 5 | 0.5 ± 0.1 |

The L-enantiomer [(−)-10] is revealed to be a slow onset tight binding inhibitor of the PNPs of human, bovine and Plasmodium falciparum (the protozoan parasite responsible for malaria) origins. It shows surprising potency in the above assays.

Although the invention has been described by way of example, it should be appreciated that variations or modifications may be made without departing from the scope of the invention. Furthermore, when known equivalents exist to specific features, such equivalents are incorporated as if specifically referred to in the specification.

### INDUSTRIAL APPLICABILITY

The invention relates to compounds which are the L-enantiomeric forms of nucleoside analogues. These compounds are expected to be useful as pharmaceuticals in the treatment of certain diseases such as cancer, bacterial infection, parasitic infection, and T-cell mediated diseases.

1. A compound of formula (I): 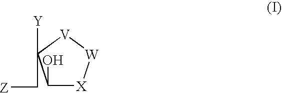
wherein:
V is selected from CH₂ and NH, and W is selected from NR¹ and NR²; or V is selected from NR¹ and NR², and W is selected from CH₂ and NH;
X is selected from CH₂ and CHOH in the R or S-configuration;
Y is selected from hydrogen, halogen and hydroxy, except where V is selected from NH, NR¹ and NR² then Y is hydrogen;
Z is selected from hydrogen, halogen, hydroxy, SQ, OQ and Q, where Q is an optionally substituted alkyl, aralkyl or aryl group;
R¹ is a radical of the formula (II) 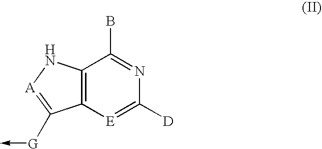
R² is a radical of the formula (III) 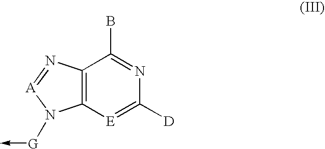
A is selected from N, CH and CR, where R is selected from halogen, optionally substituted alkyl, aralkyl or aryl, OH, NH₂, NHR³, NR³R⁴ and SR⁵, where R³, R⁴ and R⁵ are each optionally substituted alkyl, aralkyl or aryl groups;
B is selected from OH, NH₂, NHR⁶, SH, hydrogen and halogen, where R⁶ is an optionally substituted alkyl, aralkyl or aryl group;
D is selected from OH, NH₂, NHR⁷, hydrogen, halogen and SCH₃, where R⁷ is an optionally substituted alkyl, aralkyl or aryl group;
E is selected from N and CH;
G is selected from CH₂ and NH, or G is absent, provided that where W is NR¹ or NR² and G is NH then V is CH₂, and provided that where V is NR¹ or NR² and G is NH then W is CH₂;
or a tautomer thereof, or a pharmaceutically acceptable salt thereof, or an ester thereof, or a prodrug thereof.

2. A compound as claimed in claim 1 where G is CH₂.

3. A compound as claimed in claim 1 where V is CH₂.

4. A compound as claimed in claim 1 where X is CH₂.

5. A compound as claimed in claim 1 where Z is hydrogen, halogen, hydroxy, SQ, OQ or Q.

6. A compound as claimed in claim 5 where Z is OH.

7. A compound as claimed in claim 5 where Z is SQ.

8. A compound as claimed in claim 5 where Z is Q.

9. A compound as claimed in claim 1 where, when Z is SQ, OQ or Q, Q is substituted with one or more substituents selected from OH, halogen methoxy, amino, or carboxy.

10. A compound as claimed in claim 9 where Q is substituted with one or more substituents selected from fluorine or chlorine.

11. A compound as claimed in claim 1 where W is NR¹.

12. A compound as claimed in claim 1 where W is NR².

13. A compound as claimed in claim 1 where W is NH, NR¹ or NR² and X is CH₂.

14. A compound as claimed in claim 1 where V, X and G are all CH₂, Z is OH and W is NR¹.

15. A compound as claimed in claim 1 where V, X and G are all CH₂, Z is SQ and W is NR¹.

16. A compound as claimed in claim 1 where Y is hydrogen.

17. A compound as claimed in claim 1 where Y is hydroxy.

18. A compound as claimed in claim 1 where B is hydroxy.

19. A compound as claimed in claim 1 where B is NH₂.

20. A compound as claimed in claim 1 where A is CH.

21. A compound as claimed in claim 1 where A is N.

22. A compound as claimed in claim 1 where D is H.

23. A compound as claimed in claim 1 where D is NH₂.

24. A compound as claimed in claim 1 where E is N.

25. A compound as claimed in claim 1 where any halogen is chlorine or fluorine.

26. A compound as claimed in claim 1 where any one or more of R³, R⁴, R⁵, R⁶ and R⁷ is each substituted with one or more substituents selected from OH or halogen.

27. A compound as claimed in claim 26 where the halogen is fluorine or chlorine.

28. A compound as claimed in claim 1, selected from:
(3S,4S)-1-[(9-deazaadenin-9-yl)methyl]-3-hydroxy-4-(hydroxymethyl)-pyrrolidine;
(3S,4S)-1-[(9-deazaadenin-9-yl)methyl]-3-hydroxy-4-methyl-pyrrolidine;
(3S,4S)-1-[(9-deazaadenin-9-yl)methyl]-3-hydroxy-4-(2-phenylethyl)-pyrrolidine;
(3S,4R)-1-[(9-deazaadenin-9-yl)methyl]-3-hydroxy-4-(methylthiomethyl)-pyrrolidine;
(3S,4R)-1-[(9-deazaadenin-9-yl)methyl]-3-hydroxy-4-(ethylthiomethyl)-pyrrolidine;
(3S,4R)-1-[(9-deazaadenin-9-yl)methyl]-3-hydroxy-4-(2-fluoroethylthiomethyl)-pyrrolidine;
(3S,4R)-1-[(9-deazaadenin-9-yl)methyl]-3-hydroxy-4-(2-hydroxyethylthiomethyl)-pyrrolidine;
(3S,4R)-1-[(9-deazaadenin-9-yl)methyl]-3-hydroxy-4-(propylthiomethyl)-pyrrolidine;
(3S,4R)-1-[(9-deazaadenin-9-yl)methyl]-3-hydroxy-4-(isopropylthiomethyl)-pyrrolidine;
(3S,4R)-1-[(9-deazaadenin-9-yl)methyl]-3-hydroxy-4-(butylthiomethyl)-pyrrolidine;
(3S,4R)-1-[(9-deazaadenin-9-yl)methyl]-3-hydroxy-4-(cyclohexylylthiomethyl)-pyrrolidine;
(3S,4R)-1-[(9-deazaadenin-9-yl)methyl]-3-hydroxy-4-(cyclohexylmethylthiomethyl)-pyrrolidine;
(3S,4R)-1-[(9-deazaadenin-9-yl)methyl]-3-hydroxy-4-(cyclopentylthiomethyl)-pyrrolidine;
(3S,4R)-1-[(9-deazaadenin-9-yl)methyl]-3-hydroxy-4-(phenylthiomethyl)-pyrrolidine;
(3S,4R)-1-[(9-deazaadenin-9-yl)methyl]-3-hydroxy-4-(4-fluorophenylthiomethyl)-pyrrolidine;
(3S,4R)-1-[(9-deazaadenin-9-yl)methyl]-3-hydroxy-4-(4-chlorophenylthiomethyl)-pyrrolidine;
(3S,4R)-1-[(9-deazaadenin-9-yl)methyl]-3-hydroxy-4-(3-chlorophenylthiomethyl)-pyrrolidine;
(3S,4R)-1-[(9-deazaadenin-9-yl)methyl]-3-hydroxy-4-(4-methylphenylthiomethyl)-pyrrolidine;
(3S,4R)-1-[(9-deazaadenin-9-yl)methyl]-3-hydroxy-4-(3-methylphenylthiomethyl)-pyrrolidine;
(3S,4R)-1-[(9-deazaadenin-9-yl)methyl]-3-hydroxy-4-(benzylthiomethyl)-pyrrolidine;
(3S,4S)-1-[(9-deazaadenin-9-yl)methyl]-3-acetoxy-4-(acetoxymethyl)-pyrrolidine.
(3S,4S)-1-[(9-deazaguanin-9-yl)methyl]-3-hydroxy-4-(hydroxymethyl)-pyrrolidine;
(3S,4R)-1-[(9-deazaguanin-9-yl)methyl]-3-hydroxy-4-(methylthiomethyl)-pyrrolidine;
(3S,4S)-1-[(9-deazaguanin-9-yl)methyl]-3-hydroxy-4-methyl-pyrrolidine;
(3S,4S)-1-[(9-deazahypoxanthin-9-yl)methyl]-3-hydroxy-4-(hydroxymethyl)-pyrrolidine;
(3S,4R)-1-[(9-deazahypoxanthin-9-yl)methyl]-3-hydroxy-4-(methylthiomethyl)-pyrrolidine;
(3S,4S)-1-[(9-deazahypoxanthin-9-yl)methyl]-3-hydroxy-4-methyl-pyrrolidine;
(3S,4S)-1-[(9-deaza-8-fluoro-hypoxanthin-9-yl)methyl]-3-hydroxy-4-(hydroxymethyl)-pyrrolidine;
(3R,4R)-1-[(9-deazahypoxanthin-9-yl)methyl]-3,4-dihydroxy-4-(hydroxymethyl)-pyrrolidine;
(3R,4S)-1-[(9-deazahypoxanthin-9-yl)methyl]-3,4-dihydroxy-4-(methylthiomethyl)-pyrrolidine;
(3S,4S)-1-[(9-deazaxanthin-9-yl)methyl]-3-hydroxy-4-(hydroxymethyl)-pyrrolidine;
(3S,4S)-1-[(9-deazaxanthin-9-yl)methyl]-3-hydroxy-4-(methylthiomethyl)-pyrrolidine;
(3S,4S)-1-[(6-chloro-9-deazapurin-9-yl)methyl]-3-hydroxy-4-(hydroxymethyl)-pyrrolidine;
(3S,4S)-1-[(6-azido-9-deazapurin-9-yl)methyl]-3-hydroxy-4-(hydroxymethyl)-pyrrolidine;
(3S,4S)-1-[(8-aza-9-deazaadenin-9-yl)methyl]-3-hydroxy-4-(hydroxymethyl)-pyrrolidine;
(3S,4S)-1-[(8-aza-9-deazaadenin-9-yl)methyl]-3-hydroxy-4-methyl-pyrrolidine;
(3S,4R)-1-[(8-aza-9-deazaadenin-9-yl)methyl]-3-hydroxy-4-(benzylthiomethyl)-pyrrolidine;
(3S,4R)-1-[(8-aza-9-deazaadenin-9-yl)methyl]-3-hydroxy-4-(methylthiomethyl)-pyrrolidine;
(3S,4R)-1-[(8-aza-9-deazaadenin-9-yl)methyl]-3-hydroxy-4-(ethylthiomethyl)-pyrrolidine;
(3S,4R)-1-[(8-aza-9-deazaadenin-9-yl)methyl]-3-hydroxy-4-(propylthiomethyl)-pyrrolidine;
(3S,4R)-1-[(8-aza-9-deazaadenin-9-yl)methyl]-3-hydroxy-4-(isopropylthiomethyl)-pyrrolidine;
(3S,4R)-1-[(8-aza-9-deazaadenin-9-yl)methyl]-3-hydroxy-4-(butylthiomethyl)-pyrrolidine;
(3S,4R)-1-[(8-aza-9-deazaadenin-9-yl)methyl]-3-hydroxy-4-(phenylthiomethyl)-pyrrolidine;
(3S,4R)-1-[(8-aza-9-deazaadenin-9-yl)methyl]-3-hydroxy-4-(4-fluorophenylthiomethyl)pyrrolidine;
(3S,4R)-1-[(8-aza-9-deazaadenin-9-yl)methyl]-3-hydroxy-4-(4-chlorophenylthiomethyl)-pyrrolidine;
(3S,4R)-1-[(8-aza-9-deazaadenin-9-yl)methyl]-3-hydroxy-4-(3-chlorophenylthiomethyl)-pyrrolidine;
(3S,4R)-1-[(8-aza-9-deazaadenin-9-yl)methyl]-3-hydroxy-4-(4-methylphenylthiomethyl)-pyrrolidine;
(3S,4R)-1-[(8-aza-9-deazaadenin-9-yl)methyl]-3-hydroxy-4-(3-methylphenylthiomethyl)-pyrrolidine;
(3S,4S)-1-[(8-aza-9-deazaguanin-9-yl)methyl]-3-hydroxy-4-(hydroxymethyl)-pyrrolidine;
(3S,4S)-1-[(8-aza-9-deazaguanin-9-yl)methyl]-3-hydroxy-4-methyl-pyrrolidine;
(3S,4S)-1-[(8-aza-9-deazaguanin-9-yl)methyl]-3-hydroxy-4-(methylthiomethyl)-pyrrolidine;
(3S,4S)-1-[(8-aza-9-deazahypoxanthin-9-yl)methyl]-3-hydroxy-4-(hydroxymethyl)-pyrrolidine;
(3S,4S)-1-[(8-aza-9-deazahypoxanthin-9-yl)methyl]-3-hydroxy-4-methyl-pyrrolidine;
(3S,4S)-1-[(8-aza-9-deazahypoxanthin-9-yl)methyl]-3-hydroxy-4-(methylthiomethyl)-pyrrolidine;
(3S,4S)-1-[(8-aza-9-deazaxanthin-9-yl)methyl]-3-hydroxy-4-(hydroxymethyl)-pyrrolidine; and
(3S,4S)-1-[(8-aza-9-deazaxanthin-9-yl)methyl]-3-hydroxy-4-(methylthiomethyl)-pyrrolidine.

29. A pharmaceutical composition comprising a pharmaceutically effective amount of a compound as claimed in claim 1.

30. A pharmaceutical composition comprising a pharmaceutically effective amount of a compound as claimed in claim 28.

31. A method of treating or preventing a disease or condition in which it is desirable to inhibit PNP comprising administering a pharmaceutically effective amount of a compound as claimed in claim 1 to a patient requiring treatment, where the disease or condition is cancer, a bacterial infection, a parasitic infection, or a T-cell mediated disease.

32. (canceled)

33. A method as claimed in claim 31 where the T-cell mediated disease is psoriasis, lupus, arthritis or another autoimmune disease.

34. A method as claimed in claim 31 where the parasitic infection is an infection caused by a protozoan parasite.

35. A method as claimed in claim 34 where the protozoan parasite is a parasite of the genera Giardia, Trichomonas, Leishmania, Trypanosoma, Crithidia, Herpetomonas, Leptomonas, Histomonas, Eimeria, Isopora or Plasmodium.

36. A method of immunosuppression in a patient who has undergone organ transplantation comprising administering a pharmaceutically effective amount of a compound as claimed in claim 1 to the patient.

37. A method of treating or preventing a disease or condition in which it is desirable to inhibit MTAP comprising administering a pharmaceutically effective amount of a compound as claimed in claim 1 to a patient requiring treatment, wherein the disease is cancer.

38. (canceled)

39. A method as claimed in claim 37 where the cancer is prostate cancer or head or neck tumours.

40. A method of treating or preventing a disease or condition in which it is desirable to inhibit MTAN comprising administering a pharmaceutically effective amount of a compound as claimed in claim 1 to a patient requiring treatment, wherein the disease is a bacterial infection.

41-55. (canceled)
